(19) United States
(12) Patent Application Publication
(10) Pub. No.: US 2026/0083486 A1
(43) Pub. Date: Mar. 26, 2026

(54) OSTEOSYNTHESIS PLATE AND METHOD FOR PRODUCING AN OSTEOSYNTHESIS PLATE

(71) Applicant: Karl Leibinger Asset Management GmbH & Co. KG, Mühlheim an der Donau (DE)

(72) Inventors: Martin PABST, Reichenau (DE); Martin MEER, Vöhringen (DE)

(21) Appl. No.: 19/337,007

(22) Filed: Sep. 23, 2025

(30) Foreign Application Priority Data
Sep. 25, 2024 (DE) 102024127782.3

(51) Int. Cl. A61B 17/80; A61B 17/00; A61B 90/92

(52) U.S. Cl. CPC A61B 17/8052; A61B 2017/00526; A61B 2017/00982; A61B 90/92

(57) ABSTRACT

An osteosynthesis plate (P) for fixing bone fragments incudes an elongate main body (G) with a top side (G1) and an underside (G2) opposite the top side (G1). The underside (G2) is provided for placing the osteosynthesis plate (P) on the bone fragments. The osteosynthesis plate (P) includes multiple openings (A) for accommodating one screw (SK) each. An annular insert (R) formed in one piece is arranged in at least one of the openings (A). The insert (R) includes a first and a second portion (R1, R2). The first portion (R1) faces the top side (G1) and is arranged directly adjacent to the second portion (R2) in the axial direction. The first portion (R1) has a non-rotationally symmetrical outer contour (AD1) and the second portion (R2) has a rotationally symmetrical outer contour (AD2).

### CROSS-REFERENCE TO RELATED APPLICATION

The present application is related and has right of priority to German Patent Application No. DE102024127782.3 filed on Sep. 25, 2024, which is incorporated by reference in its entirety for all purposes.

### TECHNICAL FIELD

The invention relates generally to an osteosynthesis plate for fixing bone fragments and to a method for producing such an osteosynthesis plate.

### BACKGROUND

Osteosynthesis plates are used in human and veterinary medicine to fix bone fragments, for example, after a fracture, in order to promote the growing-together of the bone fragments in an anatomically correct position.

WO 2017/139903 A1 teaches, for example, a device for bone fixation having a bone plate, which has a plate hole. A hollow cylindrical or hollow cone-shaped insert is mounted in the plate hole, which insert is provided for receiving a head of a bone screw. The insert is arranged in the plate hole in a rotationally fixed manner due to an interlocking connection, for example, by projections in the inner wall of the plate hole and corresponding indentations in the insert.

EP 2 168 513 A1 describes an osteosynthesis device having a plate that has at least one through hole. The plate has an insert around the through hole, which insert is made of a softer material than the plate. The insert and the plate are interlockingly interconnected. In order to fix the insert against rotation in relation to the plate, according to the invention, the insert is not completely rotationally symmetrical.

Approaches using such inserts, or inlays, in the plate make it possible to use a more ductile material in the region of the through holes in comparison to the rest of the body of the plate. By using the more ductile material in the region of the through holes, there is no need for a preformed internal thread, which would limit the variability of the angle of the screwing direction. The inserts, or inlays, reduce the cross-section that is available in the main body between the through holes, however, such that the number of through holes is limited.

### BRIEF SUMMARY

Example aspects of the invention provide an osteosynthesis plate with an installation space requirement for the inserts that is small. Example aspects of the invention also provide a process-safe method for producing such an osteosynthesis plate.

In example embodiments, an osteosynthesis plate for fixing bone fragments is provided. The osteosynthesis plate has an elongate main body with a top side and with an underside opposite the top side. The underside is provided for placing the osteosynthesis plate on the bone fragments. The osteosynthesis plate has multiple openings for receiving one screw each, preferably for receiving a screw head with a thread formed thereon. An annular insert formed in one piece is arranged in at least one of the openings. The insert has a first portion and a second portion, which are arranged directly adjacently to one another in the axial direction. The “axial direction” means the direction of a central axis of the insert in this case. The first portion faces the top side, such that the second portion faces the underside. The first portion has a non-rotationally symmetrical outer contour. The second portion has a rotationally symmetrical outer contour.

Due to the splitting of the insert into a first portion having a non-rotationally symmetrical outer contour and into a second portion having a rotationally symmetrical outer contour, according to example aspects of the invention, functions are distributed - the first portion is used to rotationally fix the insert relative to the main body, while the second portion is used to axially secure the insert. Due to this distribution of functions, the participating cross-sections can be optimized for the respective task, such that the installation space required for the inserts is reduced or minimized.

Preferably, the non-rotationally symmetrical outer contour of the first portion of the insert is formed by at least six protrusions, which are uniformly or non-uniformly distributed on the periphery of the insert. The term “protrusions” relates only to the shape and not to the manufacturing process. Due to the at least six protrusions, a particularly uniform distribution of force is made possible during the torque support of the insert relative to the main body of the osteosynthesis plate, such that the necessary cross-section of the first portion of the insert can be kept small. A uniform distribution of the protrusions along the periphery offers the advantage that the insert can be placed into the main body in different angular orientations, such that the installation of the osteosynthesis plate is simplified. A non-uniform distribution of the protrusions can be advantageous when a defined angular orientation of the insert during insertion into the osteosynthesis plate is desired. This is the case, for example, when a color coding having multiple colors is provided on the surface of the insert directed toward the top side. In order to orient the inserts, which have been color-coded in this way, identically to one another, a defined angular position during fitting is advantageous.

According to a preferred example embodiment, the non-rotationally symmetrical outer contour of the first portion of the insert has precisely eight or precisely nine protrusions. This number has proved, in tests, to be a good compromise between simple and reliable production, compact design, and simple installation.

Preferably, the protrusions form a larger portion of the non-rotationally symmetrical outer contour than the valleys extending between the protrusions in the circumferential direction. In other words, the valleys are narrower than the protrusions. As a result, the cross-section of the first portion of the insert is utilized particularly well.

Preferably, each of the protrusions has a circular-arc-shaped portion, the center of which is arranged coaxially with the central axis of the insert. Such a shape of the protrusions improves the distribution of force inside the protrusions during torque support.

Preferably, a ratio of the smallest diameter of the portions arranged between the protrusions and the largest diameter of the protrusions is greater than 0.8, preferably greater than 0.85, particularly preferably greater than 0.9. Due to such a ratio, the annular insert can be designed having particularly thin walls.

According to a preferred example embodiment, the non-rotationally symmetrical outer contour has an undulating shape. Preferably, the transitions extend between the wave peaks, i.e., the protrusions, and the wave valleys in the tangential direction. Such a shape of the protrusions improves the distribution of force inside the protrusions during torque support.

Preferably, a transition between the first portion and the second portion of the insert is formed by a surface that is oriented orthogonally to the central axis of the insert. This surface can be used to axially secure the insert, for example, due to the fact that the surface lies on a radially inwardly directed projection of the opening or on a step of the opening.

According to a preferred example embodiment, the opening has a radially inwardly directed projection, which has a first surface directed toward the top side of the osteosynthesis plate and a second surface directed toward the underside of the osteosynthesis plate. In this example embodiment, the second portion of the insert lies both on the first surface and on the second surface, at least in some areas. In other words, the second portion engages around the projection from both sides, such that the insert is axially secured in both axial directions.

Preferably, the insert has a rotationally symmetrical inner contour with a non-constant inner diameter. A “non-constant inner diameter” is understood, in this case, to mean that at least a portion of the inner contour has a non-constant diameter. The inner contour can have a piecewise constant diameter, although not over the entire axial extension of the inner contour.

The insert preferably has a first portion with a constant diameter. This first portion forms the smallest diameter of the inner contour. When the screw connection of the osteosynthesis plate with the bone fragments is established, the thread of the bone screw, in particular the thread formed on the screw head, molds into this first portion.

The inner contour preferably has a second portion extending from the first portion in the direction of the top side of the osteosynthesis plate. In the direction of the underside of the osteosynthesis plate, the inner contour has a third portion. The second portion and the third portion form, at least in some areas, a linearly conically shaped inner contour having a first cone angle range. In other words, the inner contour has an indentation on each of the two end faces of the first portion. As a result, the material flow that occurs when the thread molds into the first portion is improved. The first cone angle range can be, for example, between ten (10) degrees and thirty (30) degrees. The cone angle of the second portion can be the same as or different from the cone angle of the third portion.

Preferably, the inner contour has a further portion, which extends from the second portion in the direction of the top side of the osteosynthesis plate and forms, at least in some sections, a linearly conically shaped contour having a second cone angle range. Alternatively or additionally, the inner contour can have a further portion, which extends from the third portion in the direction of the underside of the osteosynthesis plate and forms, at least in some section, a linearly conically shaped contour having a second cone angle range. The second cone angle range is steeper than the first cone angle range, and is, for example, between fifty (50) degrees and eighty (80) degrees.

The opening that accommodates the insert preferably has at least one portion with a non-rotationally symmetrical inner contour. This portion is shaped such that the first portion of the insert can be accommodated in the non-rotationally symmetrical inner contour of the opening in a rotationally fixed manner.

According to an alternative example embodiment, the opening that accommodates the insert has at least one portion with a non-rotationally symmetrical inner contour, which is shaped such that the non-rotationally symmetrical outer contour of the first portion of the insert is formed when the insert is pressed into the opening.

This is realized, for example, by a tooth shape of the non-rotationally symmetrical inner-contour portion of the opening. The teeth can have a course in the direction of the top side of the osteosynthesis plate, such that a uniform molding of the non-rotationally symmetrical outer contour of the insert is achieved.

Preferably, the osteosynthesis plate has multiple round openings, wherein one insert is arranged in each of the round openings as described above. The osteosynthesis plate can also have non-round openings, for example, an oblong hole. Arranging one insert in each round opening facilitates the use of the osteosynthesis plate, since the surgeon does not need to take different instructions for using the round openings into account.

Example aspects of the invention also provide a method for producing an above-described osteosynthesis plate, which method has the following:
providing the main body and the at least one insert;
pressing in or inserting the insert in one of the openings in the main body starting from the top side of the main body; and
reshaping at least one region of the second portion of the insert for establishing an interlocking connection between the insert and the main body, starting from the underside of the main body.

Such a method makes it possible to anchor the insert in the opening in a process-safe and reliable manner.

In the step of “pressing in or inserting the insert,” an originally rotationally symmetrical region of the insert is preferably shaped to form the non-rotationally symmetrical outer contour of the insert. In other words, the non-rotationally symmetrical outer contour of the first portion of the insert first arises during the process of joining the insert into the opening. This reduces the production effort required to manufacture the insert.

According to an alternative example embodiment, the step of “providing the at least one insert” includes a step of creating the non-rotationally symmetrical outer contour of the first portion of the insert. In other words, the non-rotationally symmetrical outer contour of the insert does not first arise during the process of joining the insert into the opening, but rather already in the production of the insert itself. The non-rotationally symmetrical outer contour of the first portion of the insert can be formed, for example, by material-removing machining, by sintering, or by rolling.

The main body of the osteosynthesis plate is preferably made of a titanium alloy or a steel alloy. The insert or the inserts is/are preferably made of a titanium alloy or of pure titanium, for example, of grade 2 titanium. The main body and/or the insert or the inserts can be surface-treated, for example, anodized. The insert or the inserts can have a different surface treatment, for example, for the color labeling of different compatible screws.

### BRIEF DESCRIPTION OF THE DRAWINGS

Exemplary embodiments of the invention are described in detail on the basis of the figures. Wherein:

FIGS. 1-3 each show an isometric view of an osteosynthesis plate according to a first example embodiment;

FIG. 3b shows an isometric view of the osteosynthesis plate according to a first example embodiment with screws;

FIGS. 4-6 each show a detailed view of the osteosynthesis plate according to the first example embodiment;

FIG. 7 shows a top view of an insert according to the first example embodiment;

FIG. 8 shows a sectional view of the insert according to the first example embodiment;

FIGS. 9-10 each show a sectional view of an osteosynthesis plate with alternative example embodiments of the insert;

FIG. 11 shows a detailed sectional view of the insert according to the first example embodiment;

FIG. 12 shows a detailed view of an opening in the osteosynthesis plate according to the first example embodiment;

FIGS. 13-18 show different views of a process of joining the insert into a main body of the osteosynthesis plate according to the first example embodiment;

FIGS. 19-21 each show a detailed view of an osteosynthesis plate according to a second example embodiment; and

FIGS. 22-24 each show a sectional view of the osteosynthesis plate according to the second example embodiment.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

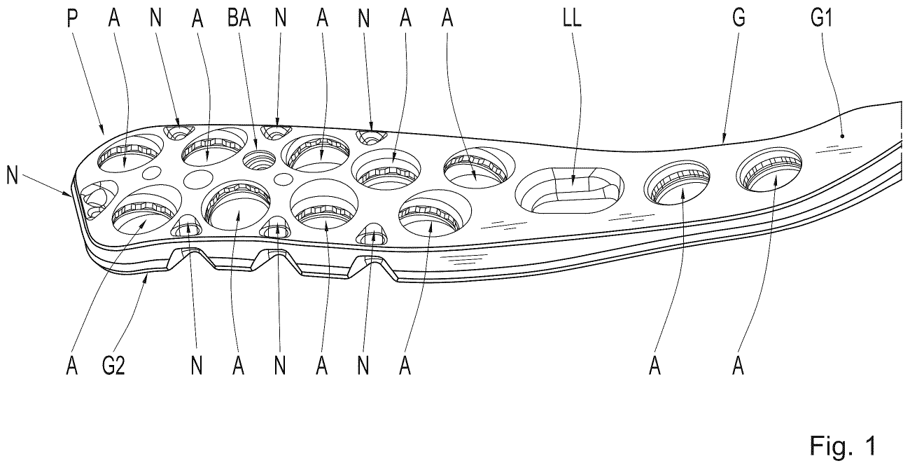

FIG. 1 shows an isometric view of an osteosynthesis plate P according to a first example embodiment. The osteosynthesis plate P is used to fix bone fragments (not shown in the figures), and is designed, by way of example, for application on the proximal humerus. The osteosynthesis plate P has an elongate main body G with a top side G1 and with an underside G2 opposite the top side G1. The underside G2 is provided for placing the osteosynthesis plate P on the bone fragments. In the representation according to FIG. 1, left is the proximal side and right is the distal side. The osteosynthesis plate P has multiple round openings A, which are designed to accommodate one screw SK each. By the screws SK, the osteosynthesis plate P can be fastened on the bone in order to fix the bone fragments in a desired position with respect to one another. Due to natural osteosynthesis, new bone material forms in the region of the gap or gaps between the bone fragments. The osteosynthesis plate P also has an oblong hole LL, which is also designed to accommodate a screw SK. The osteosynthesis plate P has multiple suture anchors N in order to be able to fasten soft tissue to the osteosynthesis plate P, for example, one or more tendons. A blind hole BA with an internal thread is provided on the top side G1 in order to be able to attach a drill guide block (not shown in FIG. 1) thereto. The main body G is made of a metal suitable for use in medical technology, for example, a titanium alloy or stainless steel.

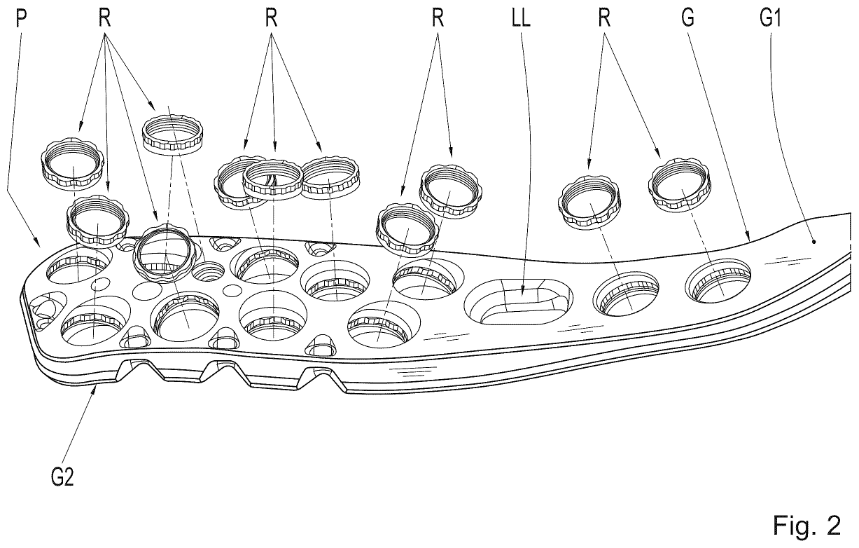

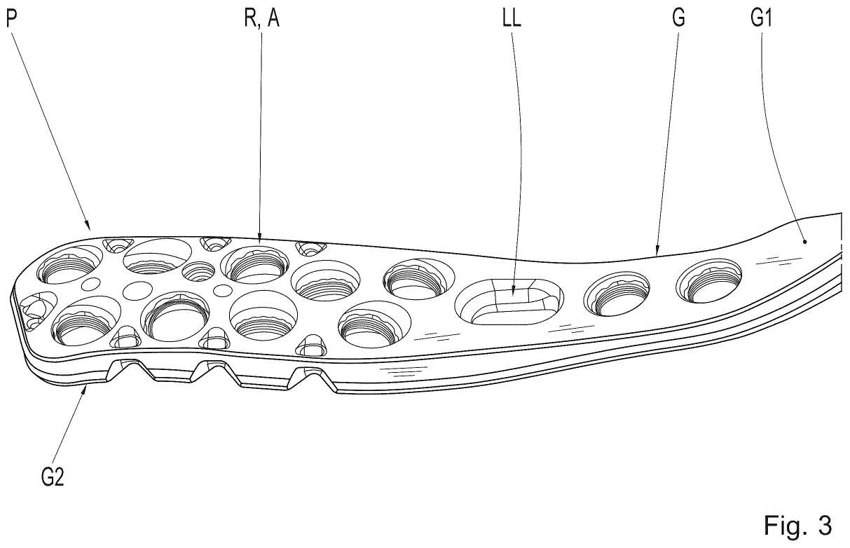

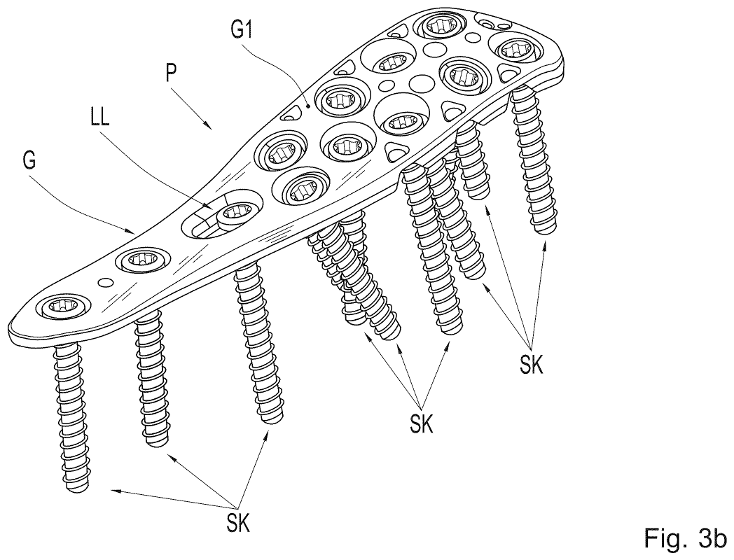

FIG. 2 shows the osteosynthesis plate P with associated inserts R. Each of the inserts R is assigned one of the round openings A. The inserts R are formed in one piece and are annular. The inserts R are made of a metal suitable for use in medical technology, which metal has a higher ductility, however, than the material of the main body G, for example, of a correspondingly ductile titanium alloy or of pure titanium. As is shown in FIG. 2, the axes of the openings A for accommodating the inserts R are differently oriented in order to provide a pre-alignment of the screws SK. The shape of the inserts R permits an orientation of the screws SK that deviates from this pre-alignment. The oblong hole LL is not associated with an insert. The oblong hole LL is designed such that an angular orientation of the screw SK associated with the oblong hole LL is defined. FIG. 3 shows the osteosynthesis plate P with inserts R arranged in the openings A.

FIG. 3b shows a further view of the osteosynthesis plate P, wherein one screw SK each is arranged in all round openings A and in the oblong hole LL. The screws SK are accommodated in the openings A and in the oblong hole LL at the screw head, which has an external thread (not visible in FIG. 3b).

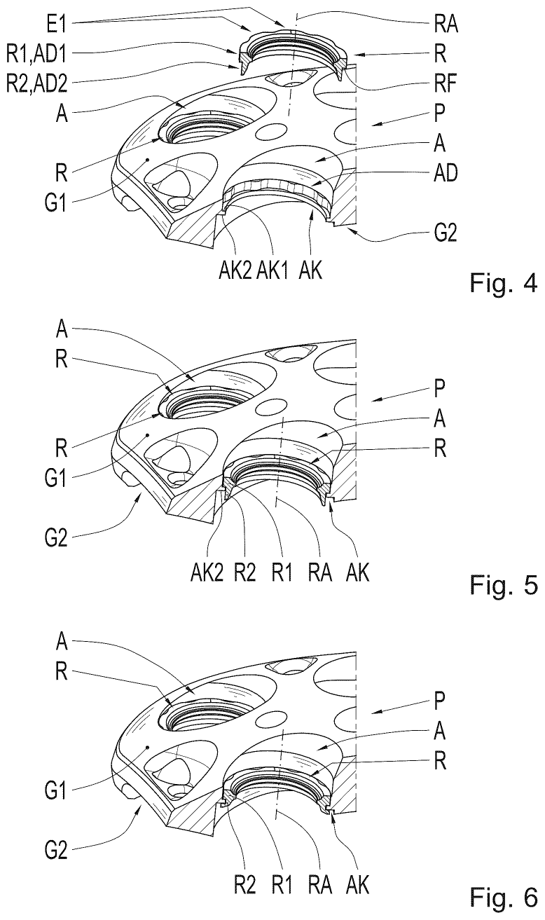

FIG. 4 shows a detailed view of the osteosynthesis plate P in an exploded view in order to visualize the accommodation of the insert R in the opening A during the process of assembling the osteosynthesis plate P. For the sake of greater clarity, parts of the main body G and of the insert R are shown as cut. The opening A has a non-rotationally symmetrical inner contour AD and a radially inwardly directed projection AK. The projection AK has a first surface AK1 directed toward the top side G1 and a second surface AK2 directed toward the underside G2. The insert R has a first portion R1 and a second portion R2. The first portion R1 faces the top side G1 and has a non-rotationally symmetrical outer contour AD1 in the form of multiple radial protrusions E1 distributed on the periphery. The second portion R2 faces the underside G2 and has a rotationally symmetrical outer contour AD2. The insert R has a surface RF, which is oriented orthogonally to the central axis RA of the insert R.

FIG. 5 shows a detailed view of the osteosynthesis plate P in which the insert R has been inserted into the opening A, such that the surface RF lies on the projection AK. The non-rotationally symmetrical outer contour AD1 of the insert R is inserted into the non-rotationally symmetrical inner contour AD of the opening A, which inner contour is formed to complement the outer contour. As a result, the insert R is rotationally fixed relative to the main body G. The second portion R2 of the insert R projects in the direction of the underside G2 beyond the second surface AK2 of the projection AK.

FIG. 6 shows a detailed view of the osteosynthesis plate P in which the insert R has been inserted into the opening A, and wherein the second portion R2 has been reshaped such that at least some portions of the second portion R2 lie on the second surface AK2, such that the projection AK is surrounded by both end faces. As a result, the insert R is axially secured relative to the main body G.

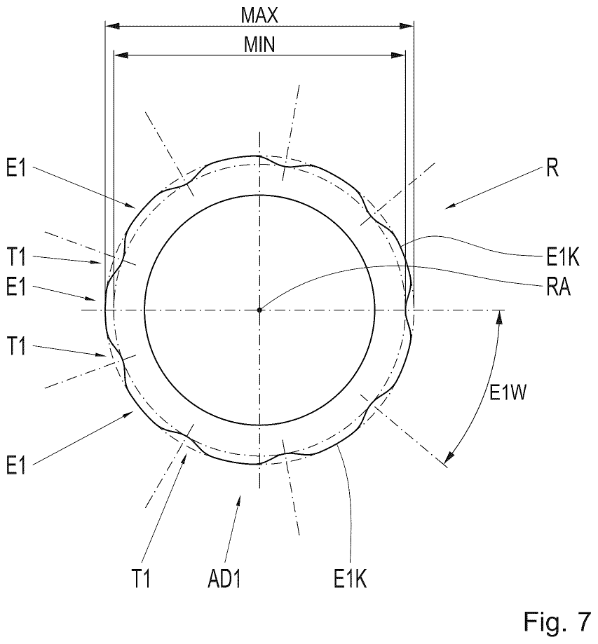

FIG. 7 shows a top view of the insert R, such that the first portion R1 of the insert R is visible. The non-rotationally symmetrical outer contour AD1 of the first portion R1 is formed by nine radial protrusions E1, which are uniformly distributed on the periphery. The non-rotationally symmetrical outer contour AD1 has an undulating shape, such that the radial protrusions E1 alternate with valleys T1. The transitions between the protrusions E1 and the valleys T1 extend in the tangential direction. The protrusions E1 form a larger portion of the non-rotationally symmetrical outer contour AD1 than the valleys T1. Each of the protrusions E1 has a circular-arc-shaped portion E1K, the center of which is arranged coaxially with the central axis RA of the insert R. The protrusions E1 are uniformly distributed on the periphery, such that each of the protrusions E1 is offset by the same angle E1W. The ratio of a smallest diameter MIN of the valleys T1 to the largest diameter MAX of the protrusions E1 is greater than 0.9.

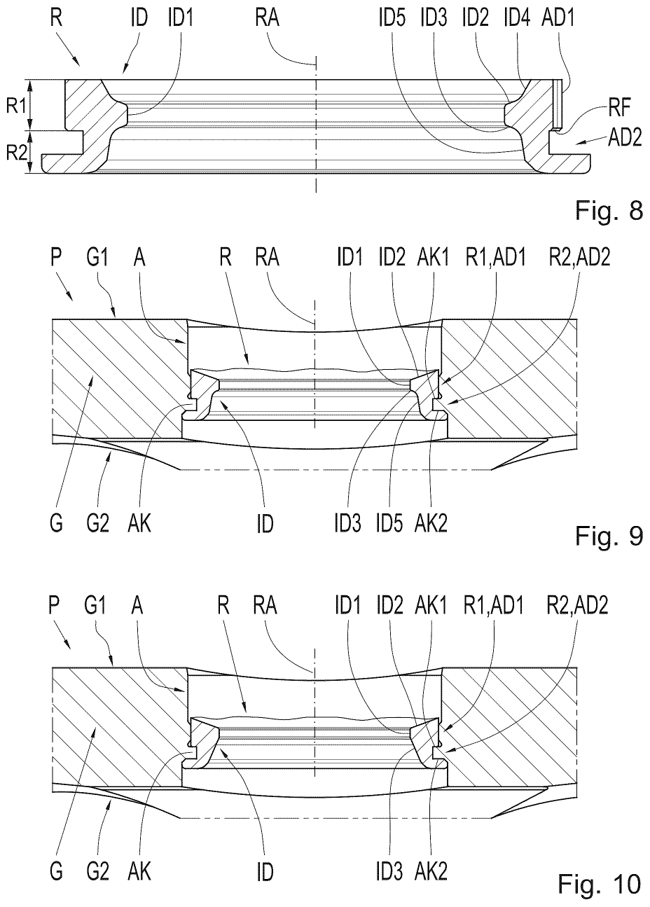

FIG. 8 shows a sectional view of the insert R, wherein the second portion R2 of the insert R has already been reshaped, as is shown in FIG. 6, in order to axially secure the insert R relative to the main body G. In FIG. 8, it is clearly apparent that the insert R has a rotationally symmetrical inner contour ID with a non-constant inner diameter. The inner contour ID has a first portion ID1 with a constant diameter, which forms the smallest diameter of the inner contour ID. When the osteosynthesis plate P is fastened to the bone fragments using the screws SK, a thread is formed in the first portion ID1 by the external thread of the screw head. The inner contour ID has a second portion ID2 extending from the first portion ID1 in the direction of the top side G1. The second portion ID2 forms a linearly conically shaped inner contour. The inner contour ID has a third portion ID3 extending from the first portion ID1 in the direction of the underside G2. The third portion ID3 forms a linearly conically shaped inner contour. The portions ID2, ID3 form a relatively flat cone angle in order to improve a flow behavior of the insert R during the formation of the thread. The inner contour ID has a fourth portion ID4 extending from the second portion ID2 in the direction of the top side G1. The fourth portion ID4 forms a linearly conically shaped inner contour. The inner contour ID has a fifth portion ID5 extending from the third portion ID3 in the direction of the underside G2. The fifth portion ID5 forms a linearly conically shaped inner contour. The inner contour ID of the insert R permits an anchoring of the screw SK at different angles relative to the central axis RA of the insert R.

FIG. 9 shows a sectional view of the osteosynthesis plate P with an alternative example embodiment of the insert R. In comparison to the example embodiment shown in FIG. 8, the insert R does not have a fourth portion ID4. Instead, the second portion ID2 extends to the end face of the insert R that is directed toward the top side G1 of the osteosynthesis plate P.

FIG. 10 shows a sectional view of the osteosynthesis plate P with a further alternative example embodiment of the insert R. In comparison to the example embodiment shown in FIG. 9, the insert R does not have a fifth portion ID5. Instead, the third portion ID3 extends to the end face of the insert R that is directed toward the underside G2 of the osteosynthesis plate P.

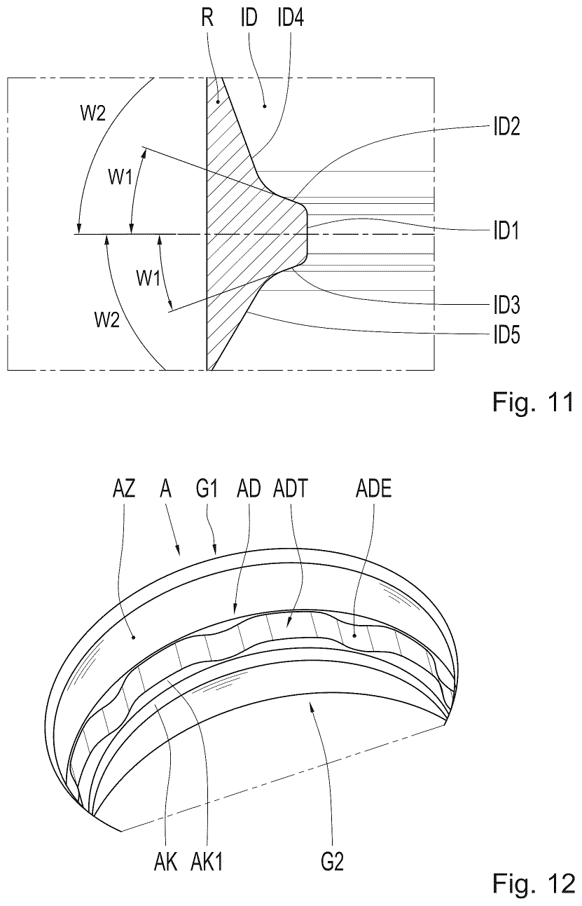

FIG. 11 shows a detailed view of the insert R to show the inner contour ID in the region of the first portion ID1. The insert R is designed as shown in FIG. 8. From the representation according to FIG. 11, it becomes clear that the conical portions ID2, ID3 directly adjacent to the first portion ID1 have a flatter cone angle range W1 than the conical portions ID4, ID5, which are adjacent thereto and have a steeper cone angle range W2.

FIG. 12 shows a detailed view of the opening A to show the non-rotationally symmetrical inner contour AD, and of the projection AK. The non-rotationally symmetrical inner contour AD has radially inwardly directed protrusions ADE, which protrude between cylindrically shaped portions ADT. Extending from the non-rotationally symmetrical inner contour AD, the opening A has a cylindrical portion AZ in the direction of the top side G1. The surface AK1 of the projection AK facing the top side G1 directly adjoins the non-rotationally symmetrical inner contour AD.

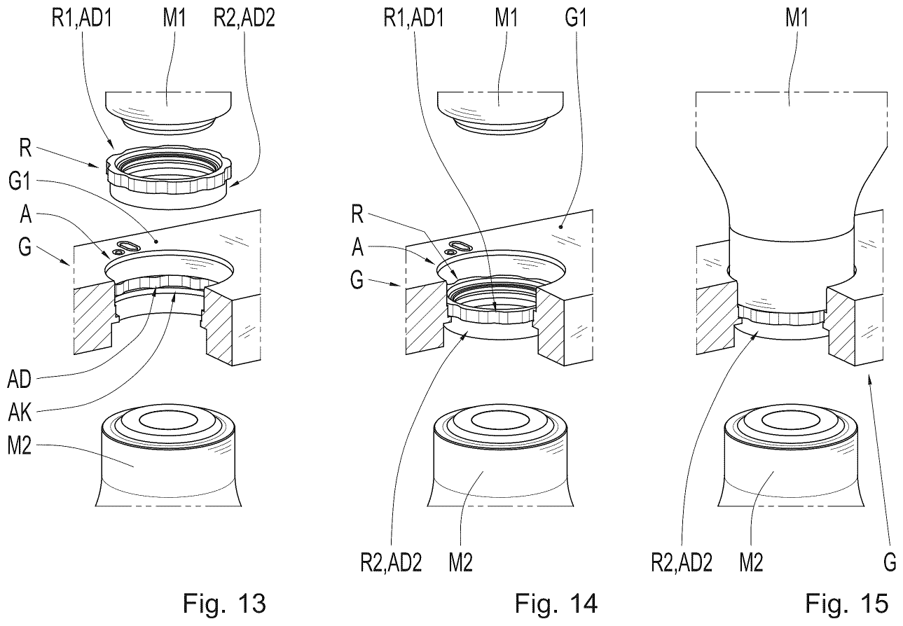

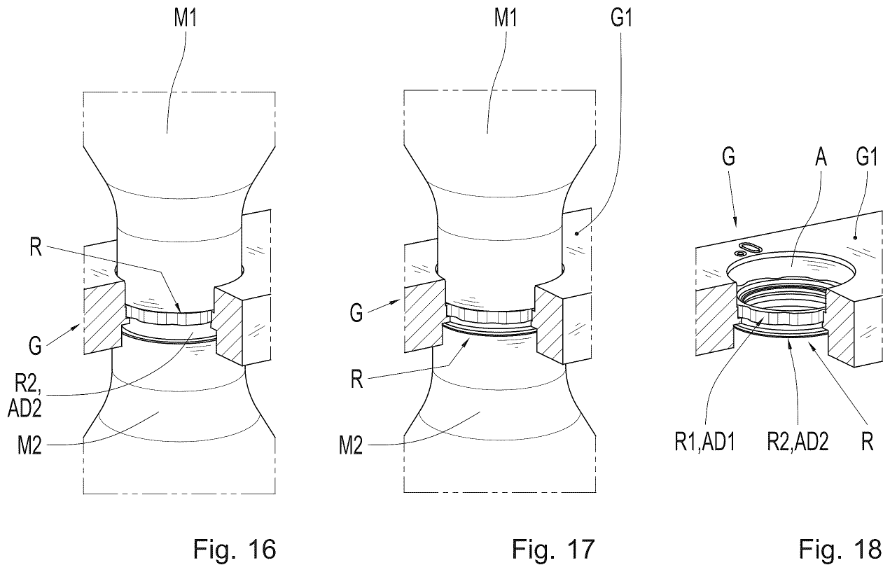

FIGS. 13 through 18 show different views of a process of joining the insert R into the main body G of the osteosynthesis plate P. In the view according to FIG. 13, the main body G and the insert R are provided between two tools M1, M2. In the view according to FIG. 14, the insert R is inserted into the opening A in the main body G, such that the non-rotationally symmetrical outer contour AD1 is accommodated in the complementary non-rotationally symmetrical inner contour AD. In the view according to FIG. 15, the insert R is pressed into the opening A by the tool M1, such that the insert R lies on the radially inwardly directed projection AK. In the view according to FIG. 16, the tool M2 is guided from the underside G2 in the direction of the second portion R2 of the insert R, such that the tool M2 lies on the insert R. In the view according to FIG. 17, the second portion R2 of the insert R is reshaped by the tool M2, such that the radially inwardly directed projection AK is surrounded by both end faces of the insert R. The insert R is held in place by the tool M1 in the direction of the top side G1. FIG. 18 shows the completely fitted insert R after the tools M1, M2 have been released.

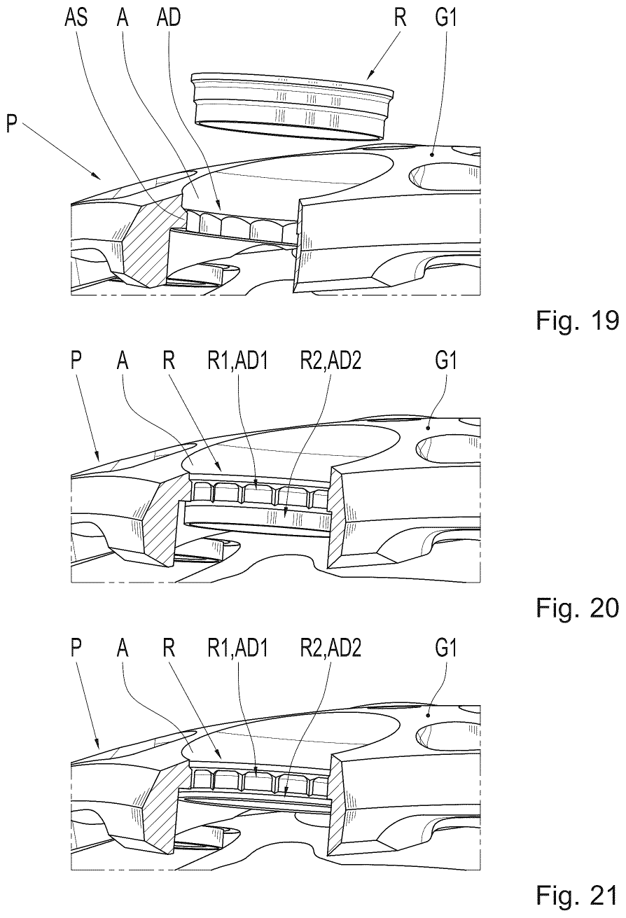

FIG. 19 shows a detailed view of an osteosynthesis plate P according to a second example embodiment. The non-rotationally symmetrical inner contour ID of the opening A has a step AS, which forms a toothing. The insert R does not yet have a rotationally symmetrical outer contour in the non-fitted state shown in FIG. 19. Instead, the insert R is rotationally symmetrical in the non-fitted state.

FIG. 20 shows a detailed view of the osteosynthesis plate P according to the second example embodiment, in which the insert R is inserted in the main body G of the osteosynthesis plate P. Due to the insertion of the insert R into the toothing of the step AS, the non-rotationally symmetrical outer contour AD1 is formed on the first portion R1 of the insert R. The second portion R2 lying spatially thereunder has a smaller diameter, such that the second portion R2 is not reshaped by the toothing of the step AS.

FIG. 21 shows a detailed view of the osteosynthesis plate P according to the second example embodiment, in which the insert R is completely fitted in the main body G of the osteosynthesis plate P. The second portion R2 is then reshaped, such that the second portion R2 surrounds a surface of the step AS directed toward the underside G2. As a result, the insert R is axially secured relative to the main body G.

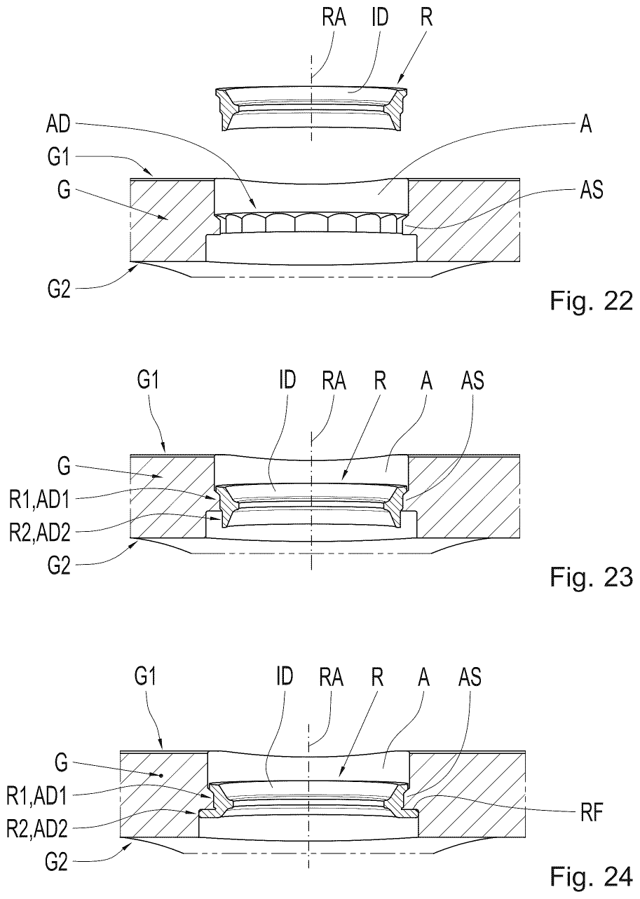

FIG. 22 through FIG. 24 show different sectional views of a portion of the osteosynthesis plate P according to the second example embodiment during the fitting of the insert R into the main body G, although without showing the tools. In the view according to FIG. 22, a state is shown, in which the insert R has not yet been fitted in the main body G. The step AS with the toothing formed thereon is clearly visible in the representation according to FIG. 22. In the view according to FIG. 23, a state is shown, in which the insert R has been inserted into the main body G, such that the non-rotationally symmetrical outer contour AD1 is formed on the first portion R1 of the insert R. In this state, the second portion R2 has not yet been reshaped. In the view according to FIG. 24, a state is shown, in which the second portion R2 of the insert R has been reshaped, such that a surface RF is formed at the transition between the first and the second portions R1, R2, which surface is oriented orthogonally to the central axis RA of the insert R and lies on an end face of the step AS facing the underside G2.

The inner contour ID of the insert R according to the second example embodiment corresponds, by way of example, to the inner contour ID of the insert R (shown in FIG. 8) according to the first example embodiment. This is shown only by way of example. The inner contour ID of the insert R according to the second example embodiment could also be designed as shown in FIG. 9 or FIG. 10.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

### LIST OF REFERENCE CHARACTERS

P osteosynthesis plate
SK screw
G main body
G1 top side
G2 underside
N suture anchor
BA blind hole
A opening
AK projection
AK1 first surface
AK2 second surface
AD non-rotationally symmetrical inner contour
ADE protrusions
ADT cylindrical portions
AZ cylindrical portion
AS step
LL oblong hole
R insert
RA central axis
R1 first portion of the insert
AD1 non-rotationally symmetrical outer contour
E1 protrusions
E1W angle
MIN smallest diameter
MAX largest diameter
E1K circular-arc-shaped portion
T1 valleys
R2 second portion of the insert
AD2 rotationally symmetrical outer contour
RF surface
ID inner contour
ID1 first portion
ID2 second portion
ID3 third portion
ID4, ID5 further portion
W1 first cone angle range
W2 second cone angle range
M1, M2 tool

1-19. canceled

20. An osteosynthesis plate (P) for fixing bone fragments, the osteosynthesis plate (P) comprising:
an elongate main body (G) with a top side (G1) and an underside (G2), the underside (G2) arranged opposite the top side (G1) on the elongate main body (G), the underside (G2) configured for placement on the bone fragments, the osteosynthesis plate (P) defining a plurality of openings (A) for accommodating one screw (SK) each; and
an annular insert (R) formed as one piece is arranged in at least one of the openings (A), the insert (R) comprising a first portion (R1) and a second portion (R2), wherein the first portion (R1) faces the top side (G1) of the elongate main body (G) and is arranged directly adjacent the second portion (R2) in an axial direction,
wherein the first portion (R1) has a non-rotationally symmetrical outer contour (AD1), and the second portion (R2) has a rotationally symmetrical outer contour (AD2).

21. The osteosynthesis plate (P) of claim 20, wherein the non-rotationally symmetrical outer contour (AD1) comprises at least six radial protrusions (E1).

22. The osteosynthesis plate (P) of claim 21, wherein the radial protrusions (E1) are distributed uniformly or non-uniformly on a periphery of the non-rotationally symmetrical outer contour (AD1).

23. The osteosynthesis plate (P) of claim 21, wherein the protrusions (E1) form a larger portion of the non-rotationally symmetrical outer contour (AD1) than valleys (T1) extending between the protrusions (E1) in a circumferential direction.

24. The osteosynthesis plate (P) of claim 21, wherein each of the protrusions (E1) comprises a circular-arc-shaped portion (E1K), the center of the circular-arc-shaped portion (E1K) arranged coaxially with a central axis (RA) of the insert (R).

25. The osteosynthesis plate (P) of claim 21, wherein a ratio between a smallest diameter (MIN) of the portions arranged between the protrusions (E1) and a largest diameter (MAX) of the protrusions (E1) is greater than 0.8.

26. The osteosynthesis plate (P) of claim 20, wherein the non-rotationally symmetrical outer contour (AD1) has an undulating shape.

27. The osteosynthesis plate (P) of claim 20, wherein a transition between the first and the second portions (R1, R2) of the insert (R) is formed by a surface (RF) that is orthogonal to a central axis (RA) of the insert (R).

28. The osteosynthesis plate (P) of claim 27, wherein the surface (RF) lies on a radially inwardly directed projection (AK) of the opening (A) or on a step (AS) of the opening (A).

29. The osteosynthesis plate (P) of claim 20, wherein:
the opening (A) comprises a radially inwardly directed projection (AK) that includes a first surface (AK1) directed toward the top side (G1) of the osteosynthesis plate (P) and a second surface (AK2) directed toward the underside (G2) of the osteosynthesis plate (P); and
the second portion (R2) of the insert (R) at least partially lies both on the first surface (AK1) and on the second surface (AK2).

30. The osteosynthesis plate (P) of claim 20, wherein the insert (R) comprises a rotationally symmetrical inner contour (ID) with a non-constant inner diameter.

31. The osteosynthesis plate (P) of claim 30, wherein the inner contour (ID) of the insert (R) comprises a first portion (ID1) having a constant diameter that forms a smallest diameter of the inner contour (ID).

32. The osteosynthesis plate (P) of claim 31, wherein the inner contour (ID) comprises:
a second portion (ID2) extending from the first portion (ID1) towards the top side (G1) of the osteosynthesis plate (P); and
a third portion (ID3) extending from the first portion towards the underside (G2) of the osteosynthesis plate (P), and
wherein the second and the third portions (ID2, ID3) at least partially form a linearly conically shaped inner contour having a first cone angle range (W1).

33. The osteosynthesis plate (P) of claim 32, wherein:
the inner contour (ID) comprises a further portion (ID4, ID5) extending from the second portion (ID2) towards the top side (G1) and/or from the third portion (ID3) towards the underside (G2); and
the further portion at least partially forms a linearly conically shaped contour having a second cone angle range (W2), and the second cone angle range (W2) is steeper than the first cone angle range (W1).

34. The osteosynthesis plate (P) of claim 20, wherein the opening (A) that accommodates the insert (R) comprises at least one portion having a non-rotationally symmetrical inner contour (AD) that is shaped such that the first portion (R1) of the insert (R) is accommodatable in the non-rotationally symmetrical inner contour (AD) of the opening (A) in a rotationally fixed manner.

35. The osteosynthesis plate (P) of claim 20, wherein the opening (A) that accommodates the insert (R) comprises a portion having a non-rotationally symmetrical inner contour (AD) that is shaped such that the first portion (R1) of the insert (R) is shapeable during the process of pressing the insert (R) into the opening (A).

36. The osteosynthesis plate (P) of claim 20, wherein the annular insert (R) is one of a plurality of annular inserts (R), and each of the plurality of annular inserts (R) is arranged in a respective one of the plurality of openings (A).

37. A method for manufacturing the osteosynthesis plate (P) of claim 20, comprising:
providing the main body (G) and the at least one insert (R);
pressing or inserting the insert (R) in one of the openings (A) in the main body (G) starting from the top side (G1) of the main body (G); and
reshaping at least one region of the second portion (R2) of the insert (R) for establishing an interlocking connection between the insert (R) and the main body (G), starting from the underside (G2) of the main body (G).

38. The method for manufacturing the osteosynthesis plate (P) of claim 37, wherein pressing in or inserting the insert (R) comprising shaping an originally rotationally symmetrical region of the insert (R) to form the non-rotationally symmetrical outer contour (AD1) of the insert (R).

39. The method for manufacturing the osteosynthesis plate (P) of claim 37, wherein providing the at least one insert (G) comprises creating the non-rotationally symmetrical outer contour (AD1) of the first portion (R1) of the insert (R).
